(19) United States
(12) Patent Application Publication
(10) Pub. No.: US 2024/0277622 A1
(43) Pub. Date: Aug. 22, 2024

(54) DOSING CAPSULE MADE FROM CANNABIS-DERIVED RESIN AND METHODS OF MAKING THE SAME

(71) Applicant: CAPHEADS LLC, San Diego, CA (US)

(72) Inventor: Ori LIEBERMAN, San Diego, CA (US)

(21) Appl. No.: 18/021,367

(22) PCT Filed: Aug. 5, 2021
(86) PCT No.: PCT/US2021/044732

§ 371 Date: Feb. 14, 2023

Related U.S. Application Data
(60) Provisional application No. 63/066,027, filed on Aug. 14, 2020.

(51) Int. Cl. A61K 9/48; A61K 31/047; A61K 31/192; A61K 31/352

(52) U.S. Cl. CPC A61K 9/4816; A61K 9/4875; A61K 31/047; A61K 31/192; A61K 31/352

(57) ABSTRACT

A dosing capsule has a shell, a seal and an inner material. The outer shell is made of a cannabis-derived resin and the inner material is made of an ingestible material. The seal may be made of a cannabis-derived resin or other sealing material. The outer shell and seal substantially protect the inner material from degradation and when the capsule is heated to a pre-determined temperature, the outer shell and seal liquefy, thereby allowing the inner material to be ingested.

### CROSS-REFERENCE TO RELATED APPLICATION

This application claims priority to U.S. Provisional Patent Application, Ser. No. 63/066,027 titled “Dosing Capsule Made From Cannabis-Derived Resin And Methods Of Making The Same”, filed Aug. 14, 2020, the disclosure of which is incorporated by reference herein in its entirety.

### FIELD OF THE INVENTION

This invention generally relates to methods for creating a dosing capsule made from cannabis-derived resin. The resin may have a known or pre-determined potency of THC and/or CBD. The invention incorporates a capsule made of a shell made of a cannabis-derived resin that has been hardened or crystallized. The capsule may also have a seal made of a cannabis-derived resin or other material. The shell and seal may each have a pre-determined volume and potency. The inner material may be ingestible and also have a pre-determined volume and potency, thus providing a dosing capsule with a known dose of cannabinoids.

### BACKGROUND OF THE INVENTION

THC content differs across different cannabis products, derivatives, varieties and strains. Traditionally, consumers of cannabis in products that vaporize various forms of cannabis concentrates (ie: “dabbing”) have had to rely on imprecise “estimates” of THC content in the marijuana products that they consume, and no systems are in place that satisfactorily provide cannabis consumers a reasonable assurance of the THC content they are ingesting.

Accordingly, there exists a need for products that provide accurate and known measures of the THC content for cannabis products that a consumer can ingest or consume through vaporization or by smoking or that a consumer uses to make into an edible product. More particularly, there is a need for more accurately measuring the THC content of cannabis products for such applications or uses.

Existing dosing methods for the above types of applications (vaporizing and smoking, as well as consumer made edible products) are not efficient and may create risk for the consumer. For example, ceramics are currently used as dosing discs, and a user must utilize a “dabbing tool” or tweezers to place the disc onto a hot surface where the heat vaporizes the concentrate contained within the disc. However, the ceramic portion of the disc is not consumed and therefore creates waste. Further, the heated ceramic needs to cool down before it can be removed from the device or “dab rig”, causing delays in cleanup or re-dosing. The ceramic portion of the disc may also interfere with the taste of the cannabis and may create possible health related issues. For example, the heated ceramic may burn a person's skin if touched while it is hot.

Other products use disposable tiny glass containers for dosing. However, the glass containers themselves are not consumed and must be disposed. They also create a risk of burning or cutting a person. There is also the risk that glass or glass fibers are inadvertently inhaled if the glass container breaks. Another known product involves a “pre-cartridged E-pen.” This product vibrates to let a user know they have consumed the product, but does not tell a user how much of the product has been consumed and does not control or the amount of product being dosed or consumed. In another example, users may grab some unmeasured amount of concentrate using a dabbing tool, and place the concentrate onto the hot surface of a dabbing device where the concentrate then vaporizes. This can result in the consumption of too much or too little vaporized product. Further, the inability to measure the amount of concentrate can create difficulty applying the correct amount of heat to the dabbing device or “dab rig”, which and can cause burning or create leftover residue, which will need to be cleaned. Additionally, users risk dropping the concentrate on a surface outside of the device prior to use, which may result in a loss of valuable product and/or contamination of the product due to contaminants or debris on the surface. Concentrate waste can be particularly expensive.

With respect to edibles and ingestibles created by the consumer, currently a consumer may purchase a variety of infused edibles, such as, for example, including but not limited to chocolates, gummies, candy and drinks. When adding concentrates or refined oils, the precise dosing of the content of certain ingredients, e.g., the THC content, cannot be easily determined.

There also exists a need for creating a “cleaner” means of consuming cannabis and cannabis-derived products with fewer contaminants and impurities. Currently, consumers of cannabis products using vaporizing products, such as vaporizing cartridge pens, may unknowingly inhale, ingest or consume substantial amounts of contaminants, including left over solvents used for extraction of cannabis, and the solvents may contain heavy metals, additives or added carriers, heavy metals and pesticide traces. Often contaminants become present in the cannabis due to processing techniques that take many steps and can provide opportunities for contaminants to be introduced to the cannabis.

Contaminants or impurities not only degrade the cannabis experience by leaving behind undesirable aftertastes and odors, but they also pose potential health risks for users of cannabis. The demand to reduce or eliminate such contaminants or impurities has increased in recent years, and will continue to increase as the trend toward a more health conscious user experience continues to gain momentum.

Additionally, there exists a need for creating a more shelf-stable cannabis product that does not rapidly degrade in quality, taste or THC content over time or with temperature changes. Many cannabis products or derivatives degrade with exposure to oxygen or heat, causing consumers a subdued, diluted or stale cannabis experience. For example, cannabis products commonly contain terpenes, flavonoids, THC, and other cannabinoids which evaporate, degrade or transform with exposure to oxygen, heat, moisture and UV light. Encapsulating materials such as cannabis-derived resins, concentrates, hash, flower, terpenes, flavonoids, and cannabinoids (from hemp or cannabis) helps provide the product with a longer shelf life.

Finally, within the context of smoking and vaporizing marijuana products, there exists a need to more easily dispose of and replace cannabis products after they have been consumed. Currently, cannabis users smoke or vaporize cannabis products or derivatives by using cartridges, disposable pens, or ceramic discs containing resins for their smoking experience. These products generate waste and require disposal after use. Further, products such as ceramic discs are too “hot” after use and need to be cooled down before they can be removed or else they might burn or singe consumers. As such, there exists a need for zero waste cannabis products that can be consumed during the smoking or vaporizing process.

### SUMMARY

These and other needs are addressed by the present invention, which involves an improved method of dosing within the cannabis industry whereby a capsule comprises a shell made of a cannabis-derived resin and, in some implementations, a seal. The cannabis-derived resin may be made mostly of isolated cannabinoids, such as THCA (tetrahydrocannabinol acid extracted from trichomes), CBDA (cannabidiolic acid), CBGA (cannabigerolic acid), or any other cannabinoid resin whose physical and chemical make-up allows it to crystallize or harden. Generally, cannabis-derived resins are resins derived from the trichomes of a cannabis plant.

Cannabis-derived resins include hemp-derived resins (e.g., cannabis with low levels of THC) as they also have a similar physical and chemical make-up and can crystallize as well. The cannabis-derived resin may also be derived from cannabis ruderalis.

In the present invention, cannabis-derived resins also include hemp-derived resins as they also have a similar physical and chemical make-up and can be crystallized. The cannabis-derived resin may also be made of a combination of isolated cannabinoids and other cannabinoids or terpenes that ordinarily do not crystallize or harden on their own. The capsule may also have a seal made of a cannabis-derived resin or other material.

In one aspect, the shell and seal of the capsule may be made by hardening or crystallizing a cannabis-derived resin alone or by hardening or crystallizing a mixture of different cannabis-derived resins. A cannabis-derived resin or mixture of such resins may be used to make a shell and/or seal that can encapsulate a pre-measured or pre-determined amount of an inner material. The cannabis-derived resin shell, the seal and the inner material may each have predetermined strengths, potencies and volumes, such that cannabis consumers can measure and consume precise doses of THC, THCA, CBD, CBDA or other materials.

Further, many cannabis users desire smaller doses than those currently available on the market. The present invention, due to its ability to encapsulate doses of any size shape, or potency, also allows for the creation of accurate “microdoses” (typically doses less than 0.25 grams) in order to suit the needs of users who desire a less intense cannabis experience.

Additionally, in one aspect, a feature of the present invention is better purity in comparison with cannabis products currently on the market, as the cannabis-derived resin used for the shell and seal can be formulated free from cellulose, plastics and other foreign or potentially harmful chemicals. Further, the cannabis-derived resin may be made from cannabis that is free from unnatural or artificial flavors.

One aspect of the present invention stems from the realization that there is a need to create a more shelf-stable cannabis product that does not rapidly degrade with inadvertent exposure to moisture or oxygen. Certain inner materials that can be used are concentrates that may contain volatile terpenes. The capsule prevents such volatile terpenes from evaporating as quickly as they would in an open container and helps keep the taste, smell and the experience of the inner material as intact as possible. The capsule may be comprised of a shell and seal made of a cannabis-derived resin, which itself is resistant to degradation by both heat and oxygen, and degrades at a much slower rate than most of the inner materials. It also serves as a barrier which prevents oxygen degradation or loss of potency of the inner material. The inner material may carry a full spectrum of different cannabinoids, volatile Terpenes, flavonoids, and other material.

It is an objective of the invention to reduce waste for cannabis products used in smoking or vaporizing. The capsule, including the cannabis-derived resin outer shell and seal, and the inner material, can be consumed through combustion or vaporization with little to no residual waste to dispose of. This benefit translates to a more convenient and enjoyable user experience, as there is little to no clean-up required.

Other advantages of the present invention will become readily apparent from the following detailed description. The invention is capable of other and different embodiments, and its several details are capable of modifications in various obvious respects, all without departing from the invention. Accordingly, the drawing and description are illustrative in nature, not restrictive.

### BRIEF DESCRIPTION OF THE DRAWINGS

The foregoing aspects and many of the attendant advantages of the disclosed embodiments will become more readily appreciated by reference to the following detailed description, when taken in conjunction with the accompanying drawings, wherein:

FIG. 1a is an oblique view of the dosing capsule with part of the shell surface cut away.

FIG. 1b is a side view of the dosing capsule with part of the shell surface cut away.

FIG. 2 is an isometric view of the lower portion of the preformed mold illustrating the process by which cannabis-derived resin is poured into the mold.

FIG. 3 is an isometric view of the upper portion of the preformed mold inserted into reciprocating segments (recessed portion) on the lower portion of the preformed mold containing the cannabis-derived resin.

FIG. 4 is a cross-sectional view of the preformed mold wherein the cannabis-derived resin is pressed between the upper and lower portions of the preformed mold, illustrating the process by which the mold is allowed to cool.

FIG. 5 is an isometric view of the preformed mold illustrating the process by which the mold is allowed to cool through the use of fans and cold air.

FIG. 6 illustrates the process by which the upper and lower portions of the preformed mold are separated to reveal the hardened shell of the dosing capsule.

FIG. 7 illustrates the process of pouring or inserting the inner material into the inner cavity of the dosing capsule prior to fully enclosing the outer shell of the dosing capsule.

FIG. 8a is an isometric view of the preformed mold illustrating the process of forming a cannabis-derived resin seal over the inner material to encapsulate the inner material within a capsule.

FIG. 8b shows the completed dosing capsule, with inner material fully encapsulated within a capsule, prior to being cooled and removed from the lower portion of the preformed mold.

FIG. 9a is a top view of a preformed mold having different dimensions, diameters, depths, shapes, and thicknesses within a single tray.

FIG. 9b is a lateral view of a preformed mold having different dimensions, diameters, depths, shapes, and thicknesses within a single tray.

FIG. 10 is a cross-sectional view of a dosing capsule created through 3D printing. This figure illustrates the process of 3D printing wherein the outer surface is printed nearly to completion, leaving an open channel at the top wide enough for the inner material to be inserted into the inner cavity before sealing the outer surface.

FIG. 11 is a cross-sectional view of a dosing capsule created through 3D printing, similar to that in FIG. 10, but in a different shape.

FIG. 12 is an isometric view of the lower portion of a preformed mold illustrating the process of creating a dosing capsule by filling the mold with resin in crystalline powder form.

FIG. 13 is a top view of a scale holding the lower portions of several preformed molds, illustrating the process of measuring the quantity of crystalline powder resin contained within the preformed molds.

FIG. 14 illustrates the process by which the crystalline powder resin contained within the lower portion of a preformed mold is heated and liquefied.

FIG. 15 is an isometric view of the lower portion of a preformed mold showing the liquefied resin after being heated.

FIG. 16 is an isometric view of the upper portion of the preformed mold inserted into reciprocating segments (recessed portion) on the lower portion of the preformed mold containing the liquefied resin.

FIG. 17 is a cross-sectional view of the preformed mold wherein the cannabis-derived resin is pressed between the upper and lower portions of the preformed mold, illustrating the process by which the mold is allowed to cool.

FIG. 18 illustrates the process by which the upper and lower portions of the preformed mold are separated to reveal the hardened shell of the dosing capsule.

FIG. 19 illustrates the process of pouring or inserting the inner material into the inner cavity of the dosing capsule prior to fully enclosing the outer shell of the dosing capsule.

FIG. 20 is an isometric view of the preformed mold illustrating the process of heating crystalline powder resin into a liquid and dispensing a seal over the inner material to fully encapsulate the inner material within the dosing capsule.

FIG. 21a is an isometric view of the preformed mold containing the completed dosing capsule, with inner material fully encapsulated within the capsule, prior to being removed from the preformed mold.

FIG. 21b is an oblique side view of the completed dosing capsule after being removed from the preformed mold.

FIG. 22 is an isometric view of the upper and lower portions of a preformed mold connected together to create a hollow interior with an open channel at the top.

FIG. 23 is a cross-sectional view of a dosing capsule created through one-shot injection molding. This figure illustrates the process of injection molding wherein the cannabis-derived resin and the inner material are poured simultaneously through the use of concentric needles of differing diameters through the open channel at the top of the pre-formed mold.

FIG. 24 illustrates the process by which the upper and lower portions of the preformed mold are separated to reveal the hardened shell of the dosing capsule after cooling.

FIG. 25 is an oblique view of the dosing capsule created through one-shot injection molding with part of the shell surface cut away.

FIG. 26a is an exploded view of the preformed mold illustrating the process of heating crystalline powder resin into a liquid and creating the outer shell of the dosing capsule through the use of rapid rotation.

FIG. 26b is an isometric view of the upper and lower portions of a preformed mold connected together to create a hollow interior with an open channel at the top, sealed by a removable plug.

FIG. 27 is a cross-sectional view of the preformed mold illustrating the process by which the crystalline powder resin contained within the lower portion of a preformed mold is heated and liquefied.

FIG. 28a illustrates the process by which the preformed mold containing the melted resin is tilted at various angles and rapidly rotated to allow the melted resin to coat the inner walls of the preformed mold.

FIG. 28b illustrates the process by which the preformed mold coated with melted resin is cooled as it rotates.

FIG. 29 is a cross-sectional view of the preformed mold during the cooling process showing the hardened resin shell formed against the inner walls of the preformed mold.

FIG. 30a is an isometric view of the plug being removed from the open channel at the top of the preformed mold.

FIG. 30b is an isometric view of the preformed mold illustrating the process of pouring or inserting the inner material into the inner cavity of the dosing capsule through the open channel at the top of the preformed mold.

FIG. 31a illustrates an alternative process by which the upper portion of the preformed mold is removed prior to pouring or inserting the inner material into the inner cavity of the dosing capsule.

FIG. 31b is an isometric view of the preformed mold illustrating the process of pouring or inserting the inner material into the inner cavity of the dosing capsule through the open channel in the outer shell.

FIG. 32a is an isometric view of the preformed mold illustrating the process of forming a cannabis-derived resin seal over the open channel to complete the outer shell and encapsulate the inner material within the capsule.

FIG. 32b is a side view of the completed dosing capsule after being removed from the preformed mold.

### DETAILED DESCRIPTION

A dosing capsule made from cannabis-derived resin and a method for making such a capsule is described. The capsule incorporates the use of a cannabis-derived resin in the shell and/or seal of the capsule. The capsule also contains a pre-determined dose of a second material within the capsule, such that the total dose or potency of the complete dosing capsule can be measured or known. In the following description, for the purposes of explanation, numerous specific details are set forth in order to provide a thorough understanding of the present invention. It will be apparent, however, to one skilled in the art that the present invention may be formed without these specific details.

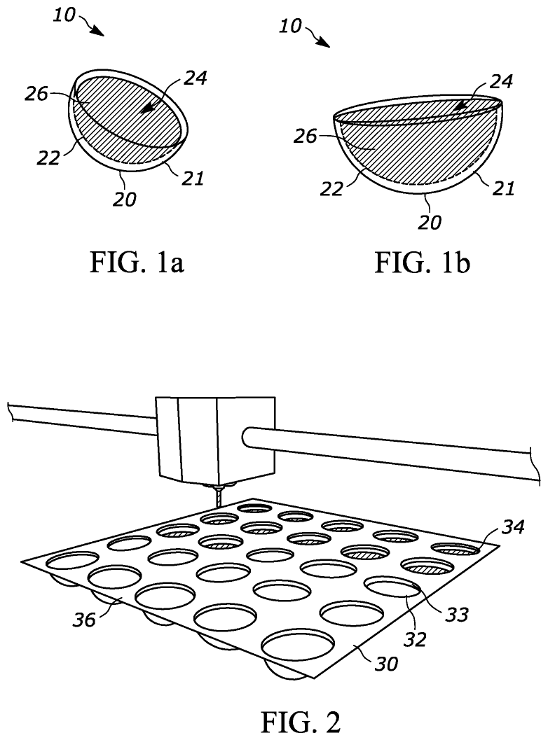

FIGS. 1-8 illustrate a dosing capsule (10) and a process of creating the dosing capsule (10) through the use of traditional or injection molding. However, the dosing capsule (10) may also be created through methods other than those described herein, such as by 3D printing, ultrasonic processes, seamless soft-gel encapsulation, or the process of thermo-forming or vacuum-forming pre-formed films made of cannabis-derived resin.

The discussion will now turn to FIG. 1a, which is an oblique view of the dosing capsule (10). The dosing capsule (10) shown in FIG. 1 includes a shell (20) with an outer surface (21), inner surface (22), and inner cavity (24). As shown, the shell (20) is generally semi-spherical shaped, with a generally spherical bottom portion and a flat top portion. The capsule (10) also has an inner material (26) contained within the shell (20). The shell (20) and the inner material (26) may have pre-measured volumes and potencies of THC and/or CBD such that the total dose of the dosing capsule (10) can be calculated. This enables cannabis users to more accurately and more confidently ingest a desired dose of THC and/or CBD or other materials.

The shell (20) may come in a variety of thicknesses in order to achieve a desired melting point and/or desired dose. The thickness of the shell (20) is customizable such that the melting point of the dosing capsule (10) can be controlled depending on whether the capsule will be orally ingested, baked, cooked, or vaporized. It is desired for the thickness of the shell to be as thin as possible for the application. For a given application, the thickness of the shell (20) may be less than 60 microns thick or greater than 600 microns thick depending on the application.

The cannabis-derived resin (34) can be extracted from any type or strain of Cannabis or hemp, including indica, sativa, and ruderalis. The extraction may be done through a solventless method or by using solvent extraction. Solventless extraction is typically done mechanically, and yields resins commonly referred to as THCA, THCA Crystalline, solventless “diamonds”, solventless THCA, fractioning THCA, or mechanical extracted THCA. Solvent extraction is typically done chemically and is often referred to as “diamond mining” or “growing diamonds”.

The cannabis-derived resin (34) may be made mostly of isolated cannabinoids, such as THCA (tetrahydrocannabinol acid extracted from trichomes), CBDA (cannabidiolic acid), CBGA (cannabigerolic acid), or any other cannabinoid resin whose physical and chemical make-up allows it to crystallize or harden. The cannabis-derived resin (34) may also be a mixture of two or more different cannabis-derived resins. The cannabis-derived resin (34) used to make the shell (20) may also be a mixture of one or more cannabis-derived resins and any other ingestible material, such as hash, terpenes or a flavor that can be vaporized. Further, the cannabis-derived resin (34) may be blended or integrated with the inner material (26) to create a homogenous dosing capsule (10) with a hardened outer shell (20). In this embodiment, crystalline cannabinoids such as THCA and CBDA are heated and blended with the inner material, which may include live resin, terpenes, terpene sauce, rosin, distillate, isolates, and other cannabis and non-cannabis materials. When blended with the inner material, the crystalline cannabinoids are used as binding agents, which allow the dosing capsule (10) to maintain its form and harden once molded.

Due to the high purity of cannabis-derived resins, the shell (20) of the dosing capsule (10) may have a predetermined volume and potency to enable accurate dosing. For example, cannabis-derived resins extracted by solvent-based or solventless methods can have a very high purity, e.g., a purity of about 98% or higher purity of THCA. The shell (20) of the dosing capsule (10) may also contain lower purities of THCA. For example, the shell (20) may be comprised of just 86.2% THCA with the remaining material consisting of terpenes and other substances. The shell (20) may come in various sizes and thicknesses. The cannabis-derived resin (34) will have a known THC, THCA and/or CBD content, the volume of the shell and seal made from the cannabis-derived resin will be known, and the content and volume of the shell's inner material will be known, so the dosing capsule (10) will have a known dose of THC, THCA and/or CBD (based on size, thickness, etc. of the shell) and a known dose of material within the shell (based on the volume created by the shell). Thus, the new dosing capsule (10) allows for an accurate and consistent dosing due to the capsule's known cannabis (e.g., THC and/or CBD) content, paired with the encapsulated inner material of a known pre-determined strength and quantity.

As shown in FIGS. 2-8, the dosing capsule (10) can be formed through the process of molding wherein the shell (20) is created by pouring a liquefied or semi-melted or gelatinous cannabis-derived resin (34) into a preformed mold (30). The preformed mold (30) may come as a tray, sheet, roll or stand-alone mold, and may include a variety of sizes, shapes, diameters, and thicknesses. The preformed mold (30) has an upper portion (38) and a lower portion (36).

As shown in FIG. 2, the cannabis-derived resin (34) is heated and poured into a recessed portion (32) of the preformed mold (30) such that it sits against inner walls (33) of the preformed mold (30). The process of pouring cannabis-derived resin (34) into the preformed mold (30) can be done manually or through the use of automatic or semi-automatic systems or processes. The amount of cannabis-derived resin (34) poured, printed, injected or otherwise dispensed can be measured as it is dispensed, and the measured amounts can be stored in a system. This data can be used for various purposes, including dose calculation, inventory tracking, supply re-ordering, and charging customers by quantity.

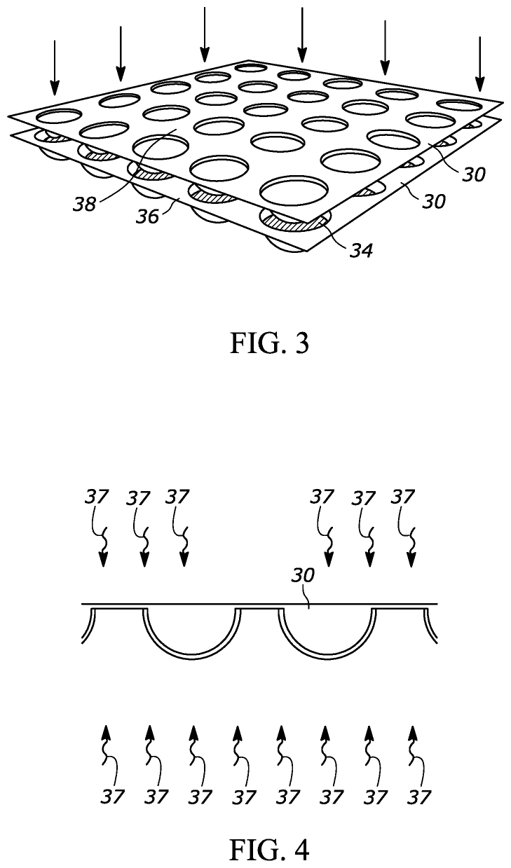

As shown in FIG. 3, the upper portion (38) of the preformed mold (30) is pressed into the lower portion (36) so that the recessed portions (32) are pressed together with reciprocating parts of the upper portion (38) of the preformed mold (30), causing the cannabis-derived-resin (34) to fill the inner walls (33) of the recessed portion (32) thereby assuming the shape of said recessed portion (32).

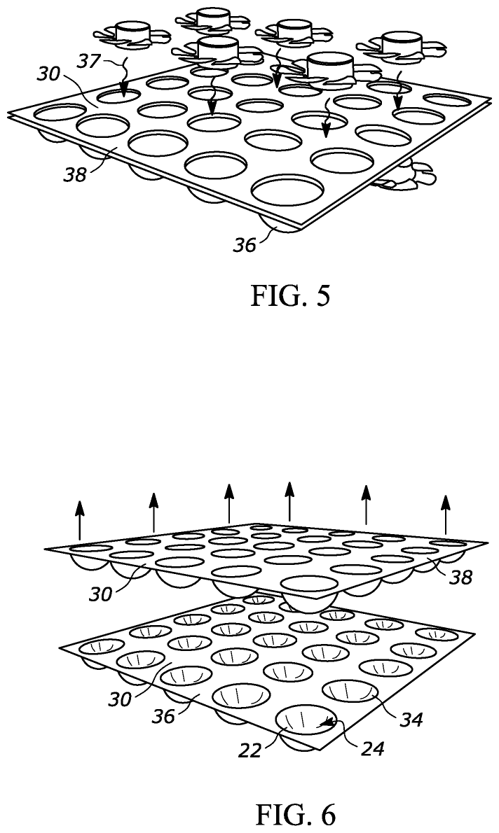

As shown in FIGS. 4-5, the preformed mold (30) containing the cannabis-derived resin (34) is allowed to cool after being pressed together. The cooling step may involve the use of cool air (37) circulated over the preformed mold (30), as indicated by the wavy arrows in FIGS. 4-5, but may also involve refrigeration or the mere passage of time. The cooled resin (34) hardens and forms a hardened shell (20).

As shown in FIG. 6, the upper portion (38) of the preformed mold (30) is removed from the lower portion (36) after the cooling step to reveal the hardened shell (20) made from cannabis-derived resin (34) that has taken the shape of the inner walls (33) of the recessed portion (32) of the preformed mold (30). Although the preformed mold (30) depicted in FIG. 6 has a semi-spherical shape, the mold (and therefore the outer shell) may be another shape, including a basic shape like a square or sphere, or a more complex shape such as a figurines.

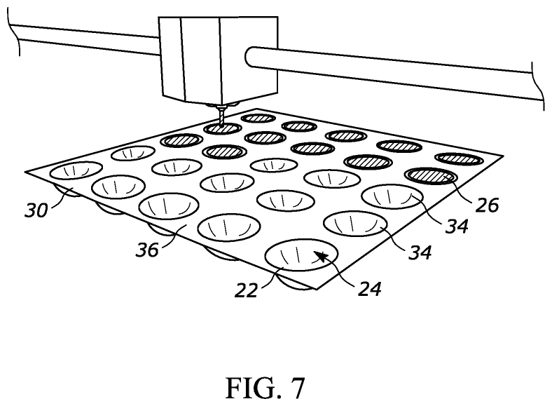

As shown in FIG. 7, the inner cavity (24) of the shell (20) is then filled with the inner material (26). The inner material (26) may be cannabis or a cannabis byproduct, including cannabis-derived resins, or it may be a non-cannabis material. For example, the inner material (26) may contain marijuana concentrates, rosin, resin, water hash or THCA or it may contain CBD or other non-psychoactive materials. The inner material (26) may come in liquid, gel, oil, powder, wax, or other solid form and will have a predetermined volume and potency to enable accurate dosing. The inner material (26) may also be any other ingestible material. Examples of such ingestible materials include marijuana concentrates, rosin, resin, water hash, THCA, hash, cannabis/hemp isolates, flower, oils, hemp, CBD, liquid nicotine, terpenes or a flavor that can be vaporized. Other ingestible materials may be used as the inner material as well, such as pharmaceuticals or nutraceuticals.

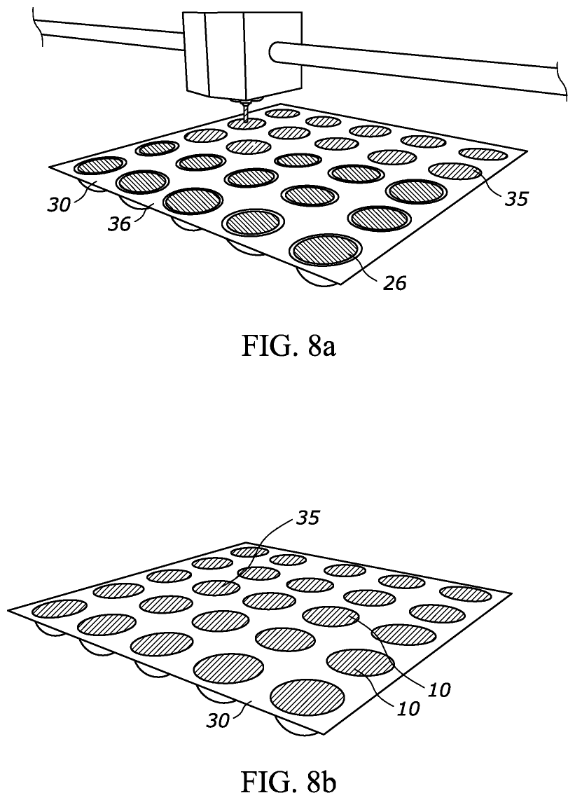

As shown in FIGS. 8a-8b, once the inner cavity (24) of the shell (20) has been filled with the inner material (26), a seal (35) may be formed or applied over the inner material (26) to encapsulate the inner material (26) within the capsule (10). The seal (35) may be any size, thickness, or diameter. Preferably, and as shown, the seal (35) is made of a cannabis-derived resin (34). However, other materials may also be used for the seal (35). If the seal (35) is not made of a cannabis-derived resin, the material used preferably has barrier properties to prevent degradation of the inner material and is also ingestible. The seal (35) may be vaporizable or non-vaporizable, such as a removable foil. Furthermore, in some examples and depending on the size of the opening to the cavity (24) of the shell (20), no seal may be used or required in some embodiments. Encapsulating the inner material (26) within a shell (20) and seal (35) prevents or slows degradation of the inner material by preventing or exposure of the inner material to air, oxygen or moisture, thus prolonging shelf life and increasing freshness of the inner material. Preferably the inner material (26) is fully encapsulated by cannabis-derived resin (34), i.e., a shell (20) and seal (35) both made of cannabis-derived resin (34), although in some embodiments, the inner material (26) may be only partly or primarily encapsulated by cannabis-derived resin (34), e.g. a shell (20) made of a cannabis-derived resin and a seal (35) made of another material that will also prevent degradation of the inner material. The consistency of the hardened cannabis-derived resin (34) is not very sticky and is also stable in ambient temperatures. This provides easy and safe handling of the capsule and dose of an inner material, which itself can be of a consistency that is too sticky to handle easily by itself.

The final dosing capsule (10) may be consumed or ingested via combustion, vaporization, and inhalation by heating the dosing capsule (10) in a smoking pipe, vaporizer, “dab rig”, or other smoking apparatus. The entire dosing capsule (10) may be dropped or inserted into a smoking apparatus, and heat will be applied to the dosing capsule (10) through the use of flame, torches, electricity, or other types of heating convection or conduction. Heat can be applied before or after the dosing capsule (10) is dropped or inserted into the smoking apparatus.

Vaporization of the dosing capsule outer shell material depends on the size of the dosing capsule, the type of resin and what it consists of. The vaporization process begins with the melting of the outer shell material at approximately 160 F-200 F. Once melted, vaporization of the entire dosing capsule, including the outer shell material and inner material, begins at approximately 300 F-500 F. For example, activation temperature ranges, i.e., the point where chemical change begins to take place for THCA starts at around 220 F-290 F and for CBDA starts at around 250 F-266 F. The boiling point, i.e., the temperature at which solids become gas, for THC starts at about 310 F-340 F and for CBD starts at about 356 F-370 F. These temperatures vary based upon pressure, chemical purity and other factors.

Once heated sufficiently, the shell (20) of the dosing capsule (10) will start to liquefy and break down, allowing the inner material (26) to be released from the inner cavity (24). Heat can be applied continuously or in stages to the entire dosing capsule (10), including both the shell (20) and the inner material (26), until all content has been fully consumed. One benefit of this invention is that both the shell (20) and the inner material (26) are consumed, leaving little to no waste or residue behind after use. The amount and duration of heat applied will depend on the desired consumption. When consumed as an edible, the dosing capsule shell (20) need only be melted. However, to consume via inhalation, the dosing capsule shell (20) must be melted and then vaporized.

The dosing capsule (10) may also be ingested as an edible. The dosing capsule (10) may be heated through conventional cooking or baking in order to liquefy and release the inner material (26) and can therefore be used in recipes for edible cannabis products such as brownies, cookies, gummies, and hard candies or to infuse other cooked items such as pastas, chicken, and fish.

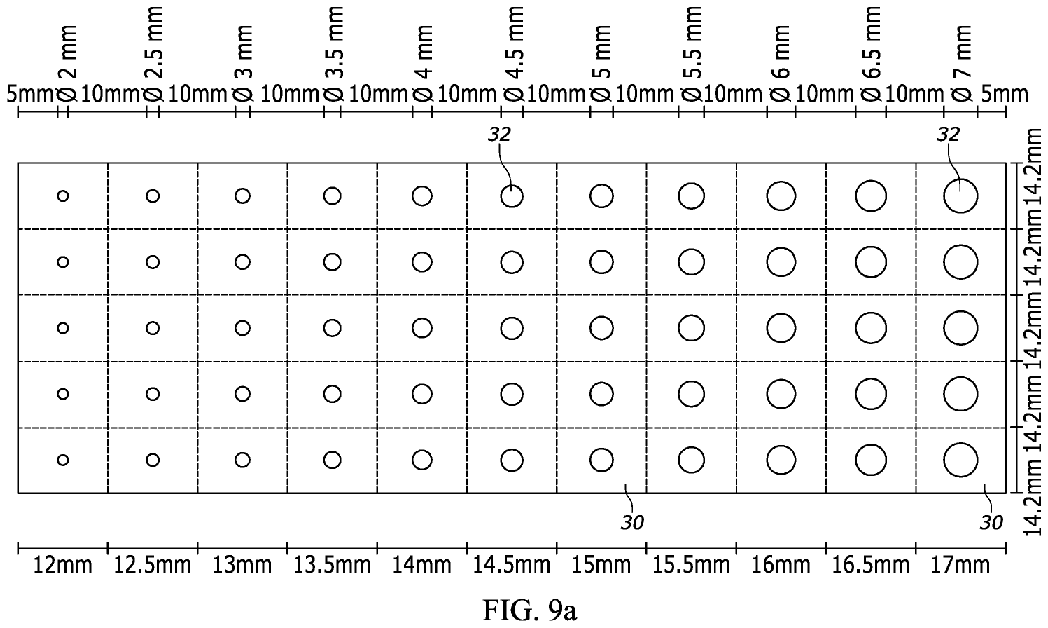

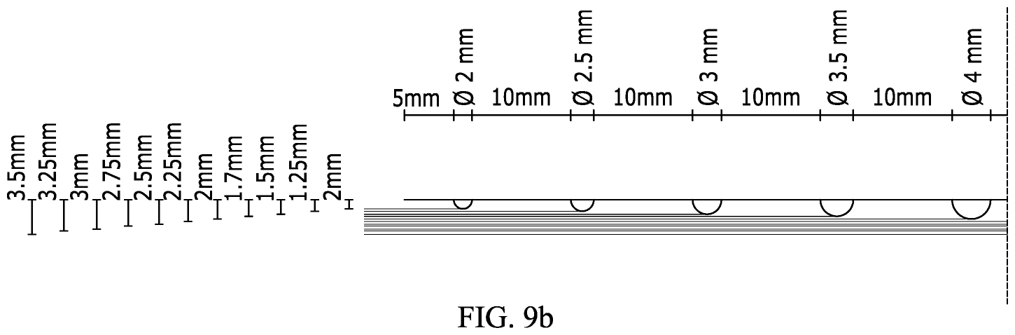

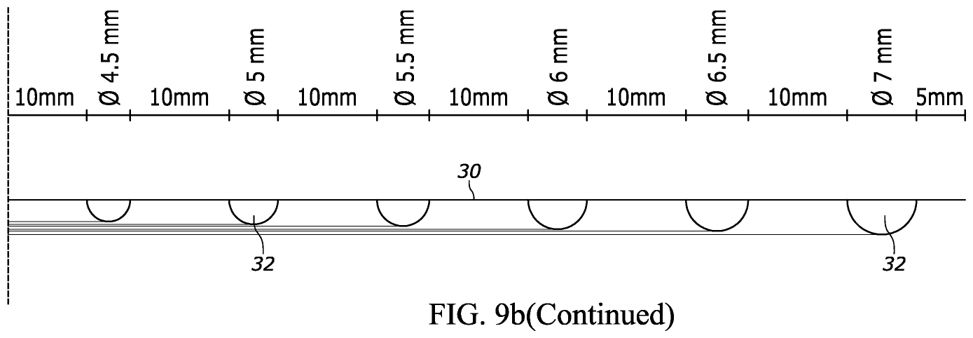

As show in FIGS. 9a-9b, the recessed portion (32) of the preformed mold (30) may be any dimension, diameter, depth, shape, and thickness. Additionally, the preformed mold (30) may contain different dimensions, diameters, depths, shapes, and thicknesses within the same tray.

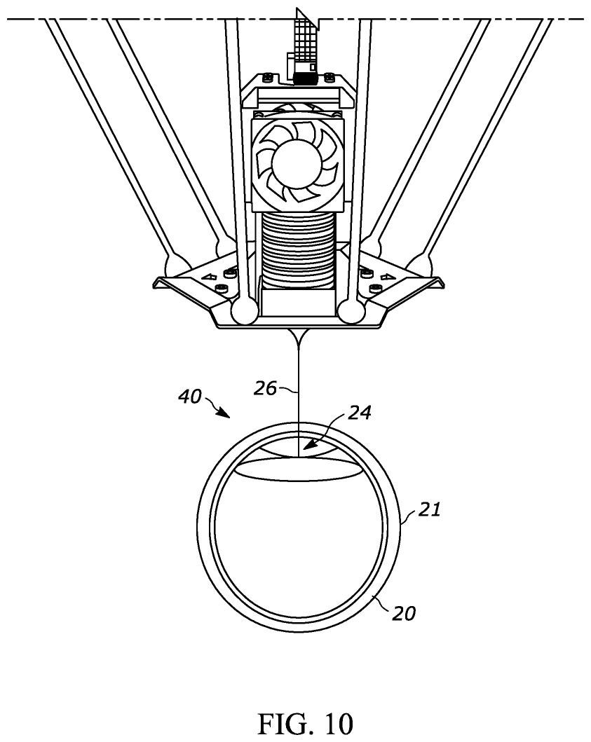

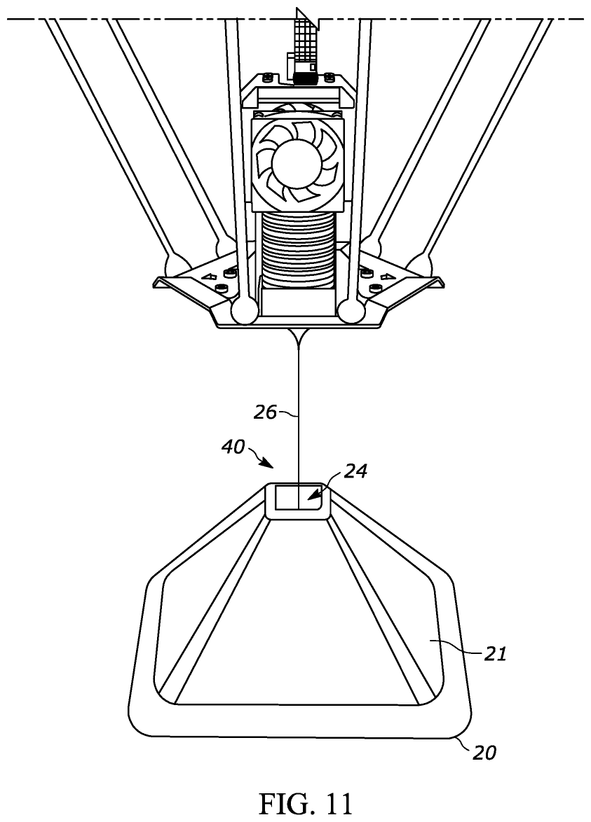

As shown in FIGS. 10-11, the dosing capsule (10) may also be created through the process of 3D printing. Here, the cannabis-derived resin (34) is heated and used to print the shell (20) nearly to completion, leaving an open channel (40) at the top wide enough for the inner material (26) to be printed, poured, or otherwise inserted into the inner cavity (24) before sealing the outer surface (21) of the shell (20) with the seal (35). The open channel (40) may be any size or diameter. The seal (35) may be a final printed layer of cannabis-derived resin (34) or other sealing material. A benefit of 3D printing the dosing capsules (10) is that the shell can take on a complex and fully customizable shape, beyond those achievable through the molding process.

As shown in FIG. 10, the dosing capsule can be 3D printed into the shape of a sphere. As shown in FIG. 11, the dosing capsule can be 3D printed into the shape of a pyramid. These shapes are for illustrative purposes only and other shapes can also be 3D printed.

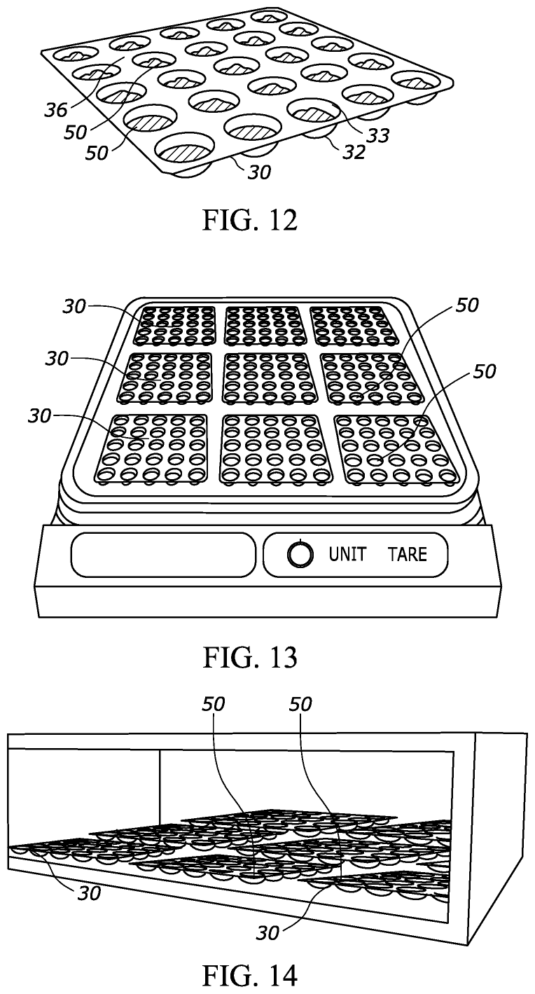

FIGS. 12-21b illustrate a dosing capsule (10) and a process of creating the dosing capsule (10) by filling the recessed portion (32) of the preformed mold (30) with cannabis-derived resin (34) in crystalline powder form and applying heat. A benefit of using cannabis-derived resin (34) in crystalline powder form (50) is that the cannabis-derived resin (34) is heated more uniformly and is less susceptible to decarboxylation and degradation when heated in this manner. In this process, the cannabis-derived resin (34) in crystalline powder form (50) preferably has a purity of about 60% or higher purity of THCA.

As shown in FIG. 12, the cannabis-derived resin (34) is pulverized into crystalline powder (50) before being added to the recessed portion (32) of the preformed mold (30). The crystalline powder (50) may be made mostly of isolated cannabinoids, such as THCA or CBDA.

As shown in FIG. 13, the preformed mold (30) can be weighed to measure the precise quantity of crystalline powder (50) contained within.

As shown in FIG. 14, the preformed mold (30) is then heated to allow the crystalline powder (50) to liquefy. The heat can be applied thorough the use of an oven, heating plate, radiant heat, convection, induction or other heating apparatus including lasers or microwaves.

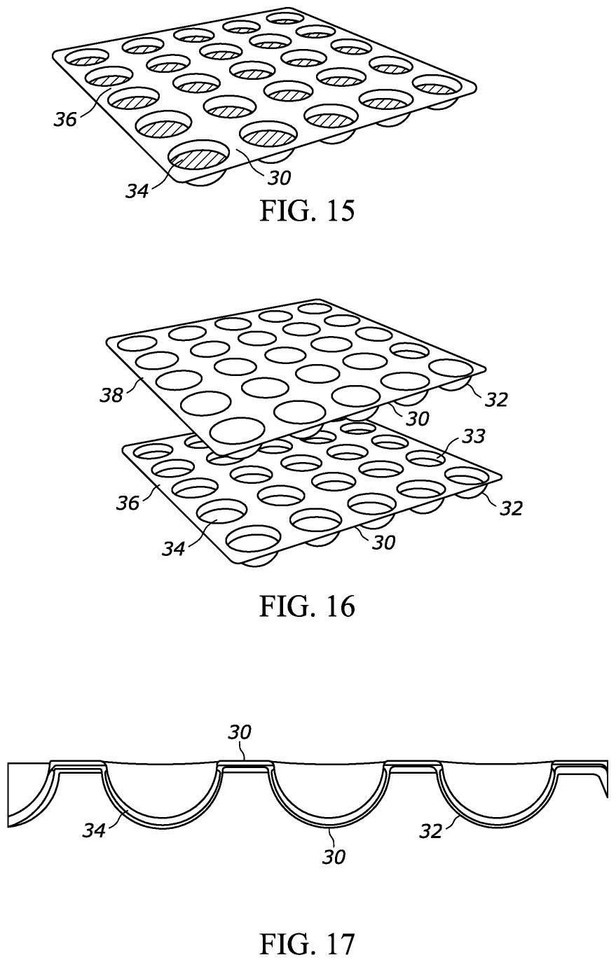

As shown in FIGS. 15-17, the preformed mold (30) is then removed from heat, and the upper portion (38) of the preformed mold (30) is pressed into the lower portion (36) so that the recessed portions (32) are pressed together with reciprocating parts of the upper portion (38) of the preformed mold (30), causing the melted crystalline powder (50) (now liquefied cannabis-derived-resin (34) to fill the inner walls (33) of the recessed portion (32) thereby assuming the shape of said recessed portion (32). The preformed mold (30) containing the cannabis-derived resin (34) is then allowed to cool after being pressed together. The cooled resin (34) hardens and forms a hardened shell (20).

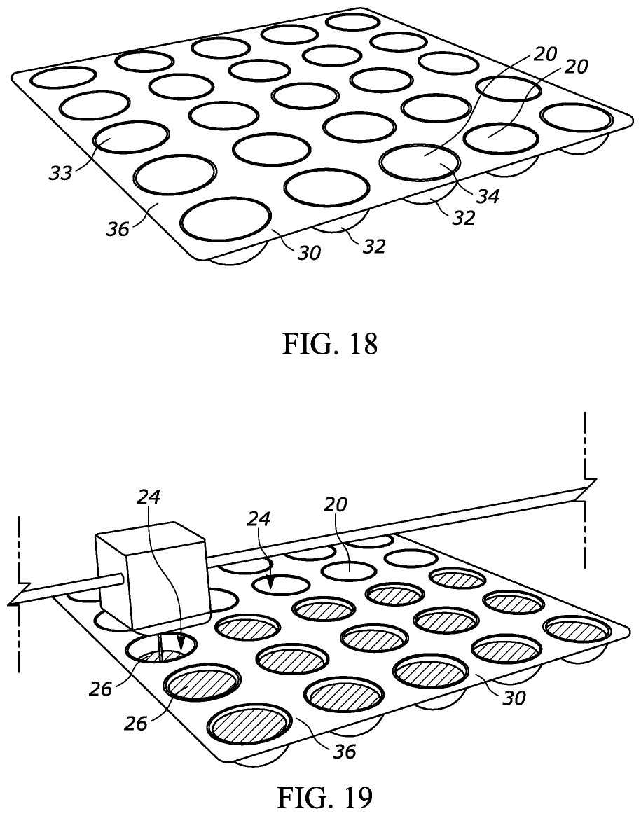

As shown in FIG. 18, the upper portion (38) of the preformed mold (30) is removed from the lower portion (36) after the cooling step to reveal the hardened shell (20) made from cannabis-derived resin (34) that has taken the shape of the inner walls (33) of the recessed portion (32) of the preformed mold (30).

As shown in FIG. 19, the inner cavity (24) of the shell (20) is then filled with the inner material (26). The inner material (26) may be cannabis or a cannabis byproduct, including cannabis-derived resins, or it may be a non-cannabis material.

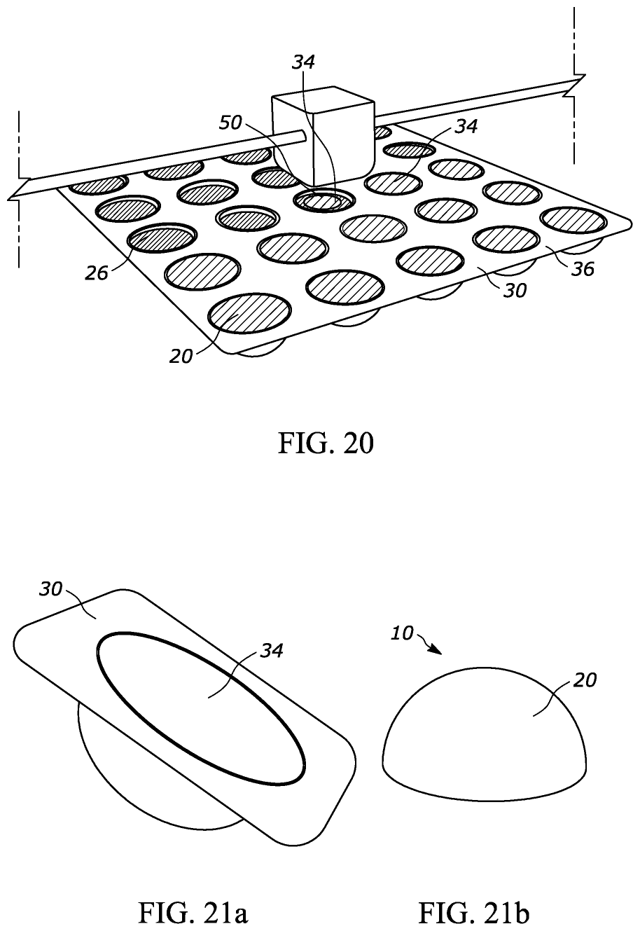

As shown in FIGS. 20-21b, more crystalline powder (50) is heated into a liquid resin (34) and poured overtop the inner material (26) to fully encapsulate the inner material (26) within the outer shell (20). The dosing capsule (10) is allowed to cool before being removed from the preformed mold (30).

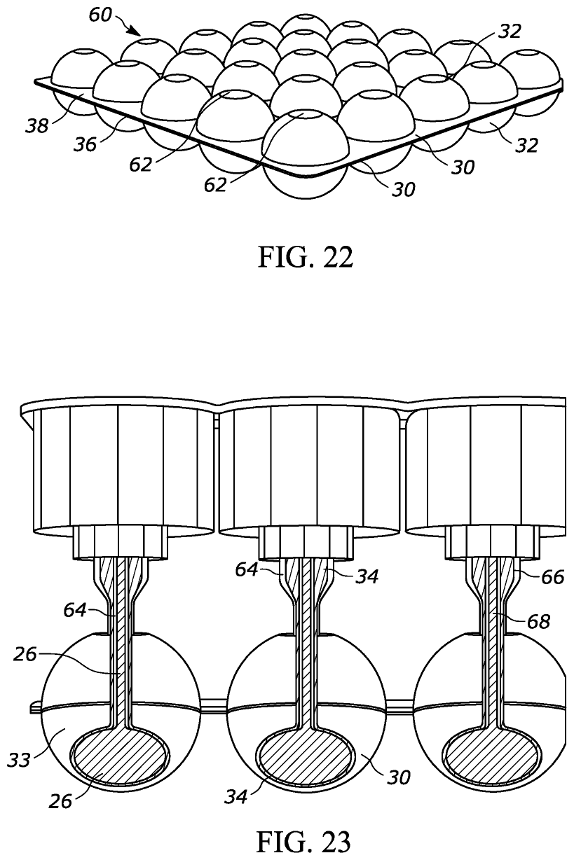

FIGS. 22-25 illustrate a dosing capsule (10) and a process of creating the dosing capsule (10) through one-shot injection molding. Here, the cannabis-derived resin (34) and the inner material (26) are injected into the open channel (60) of the preformed mold (30) simultaneously through the use of concentric injector needles (64) of differing diameters.

As shown in FIG. 22, the upper portion (38) of the preformed mold (30) is placed atop the lower portion (36) so that the recessed portions (32) are oriented away from one another and a hollow interior (62) is created.

As shown in FIG. 23, the injector needles (64) consist of a larger exterior needle (66) containing the cannabis-derived resin (34) and one or more smaller interior needles (68) containing the inner material (26). Both the exterior needle and the interior needle dispense material almost simultaneously, causing the cannabis-derived-resin (34) to fill the inner walls (33) of the preformed mold (30) thereby assuming the shape of the preformed mold (30), while the inner material (26) simultaneously fills the inside of the capsule (10).

As shown in FIG. 24, the upper portion (38) and lower portion (36) of the preformed mold (30) are allowed to cool and are then separated to reveal the hardened shell (20) of the dosing capsule (10).

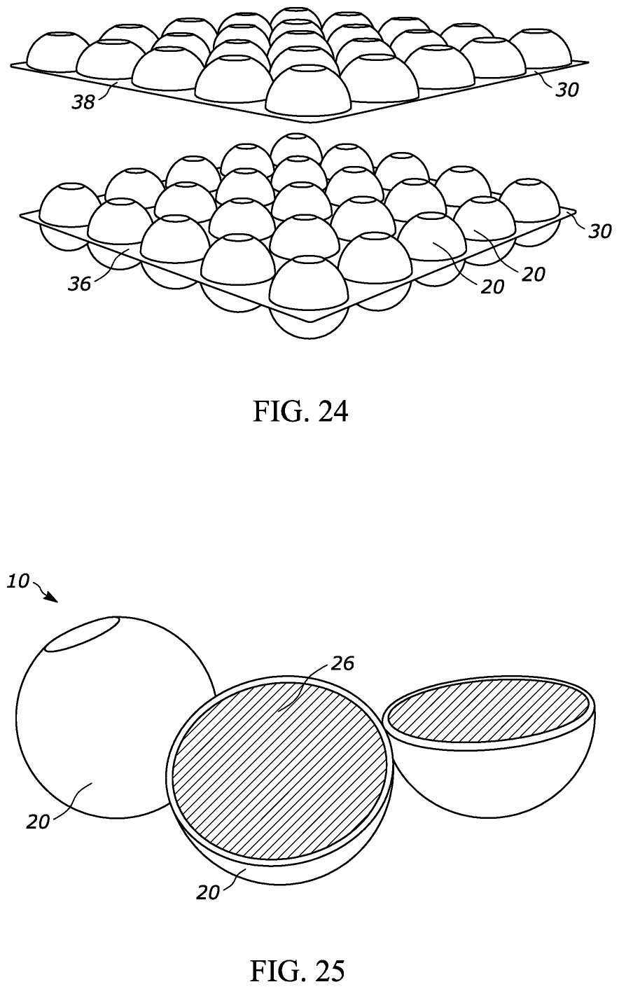

As shown in FIG. 25, the shell (20) of the dosing capsule (10) fully encapsulates the inner material (26). A benefit of forming the dosing capsules (10) through one-shot injection molding is that the outer shell (20) and inner material (26) can be poured simultaneously while still achieving complete encapsulation, a process not easily achievable with traditional injection molding.

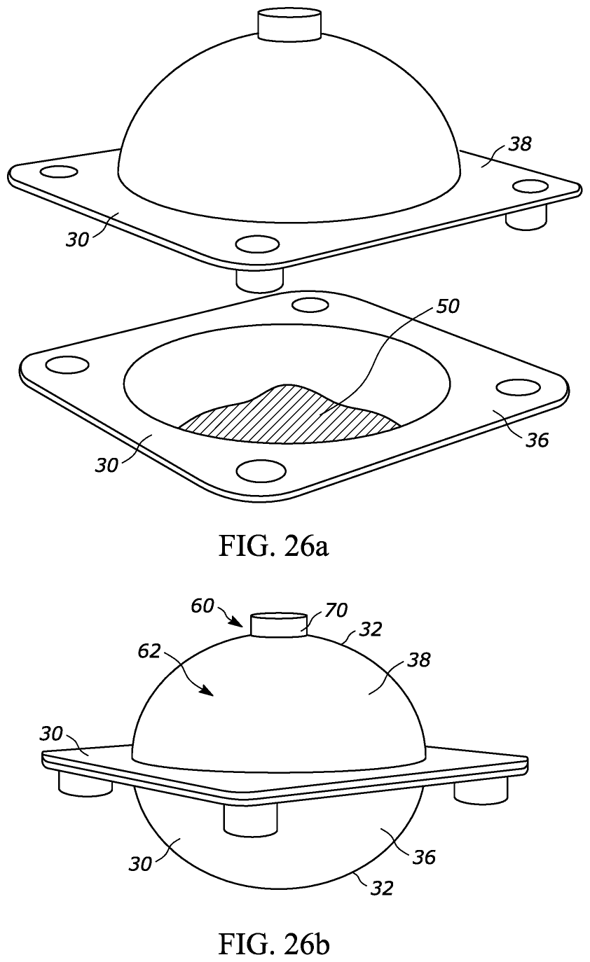

As shown in FIG. 26a, crystalline powder (50) is dispensed into the lower portion (36) of the preformed mold (30). The crystalline powder (50) may be made mostly of isolated cannabinoids, such as THCA or CBDA.

As shown in FIG. 26b, the upper portion (38) of the preformed mold (30) is placed atop the lower portion (36) so that the recessed portions (32) are oriented away from one another and a hollow interior (62) is created with an open channel (60) at the top. A plug (70) is then inserted into the open channel (60) of the preformed mold (30) to seal the preformed mold (30). The preformed mold (30) may come as a tray, sheet, roll or stand-alone mold, and may include a variety of sizes, shapes, diameters, and thicknesses.

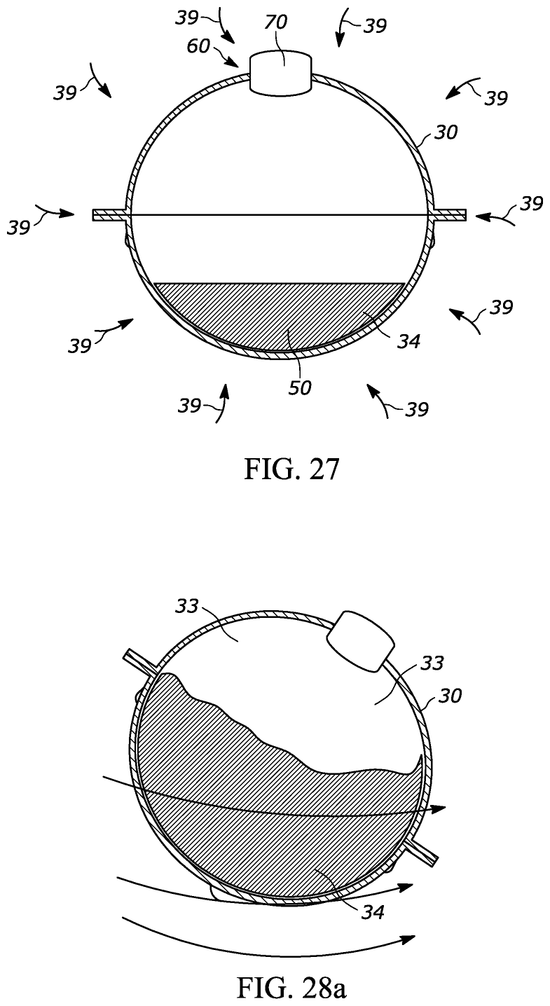

As shown in FIG. 27, heat (39) is applied to the preformed mold (30) to allow for the crystalline powder (50) to melt into a liquid resin (34). In another embodiment, pre-melted cannabis-derived resin (34) may be dispensed into the preformed mold (30) through the open channel (60). A plug (70) is then inserted into the open channel (60) of the preformed mold (30) to seal the preformed mold (30).

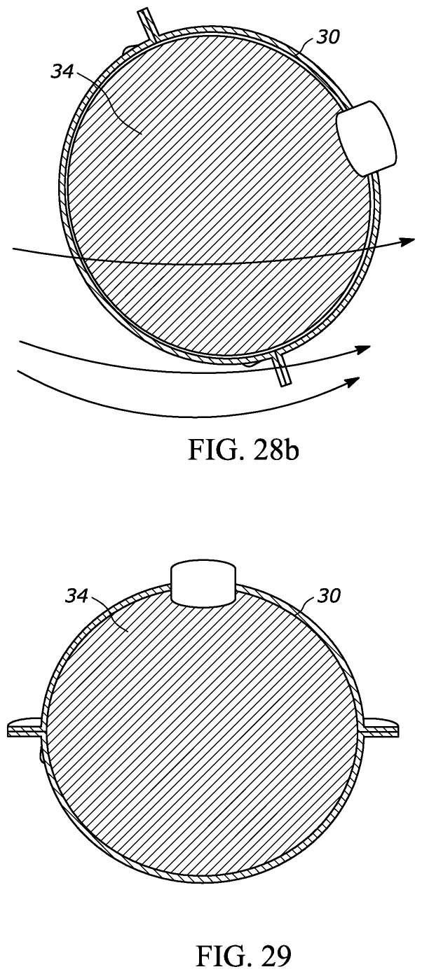

As shown in FIGS. 28a-28b, the preformed mold (30) containing the cannabis-derived resin (34) is then rapidly spun or rotated such that the cannabis-derived resin coats the inner walls (33) of the preformed mold (30). During the rotation process, the preformed mold (30) containing the cannabis-derived resin (34) may be tilted at a variety of angles, may be fully inversed, or may remain upright.

As shown in FIG. 29, the preformed mold (30) is then cooled. The cooling process may begin while the mold is being rotated, or after the mold has ceased rotating.

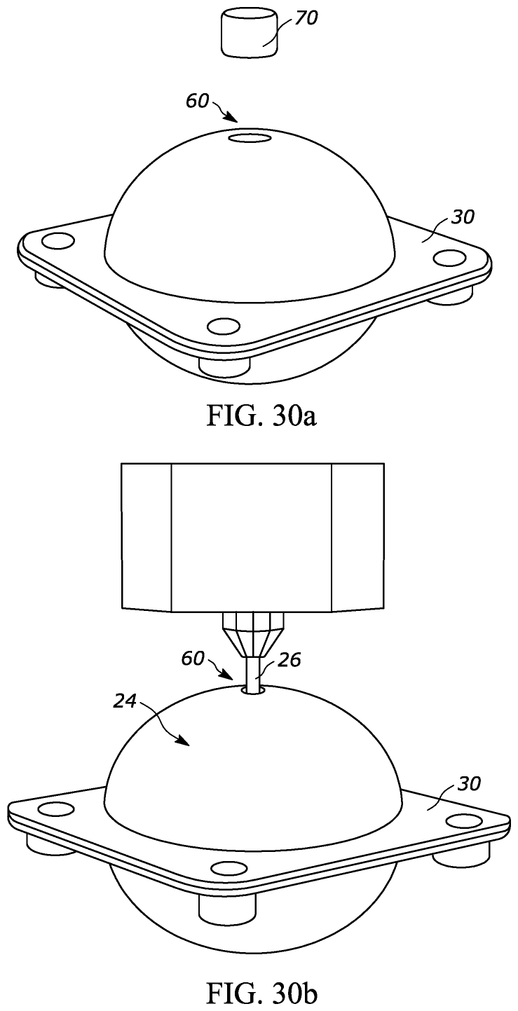

As shown in FIGS. 30a-30b, after cooling, the plug (70) is removed from the open channel (60) of the preformed mold (30) and the inner material (26) is dispensed through both the open channel (60) of the preformed mold and the open channel (40) of the outer shell (20) into the inner cavity (24) of the shell (20) and allowed to cool.

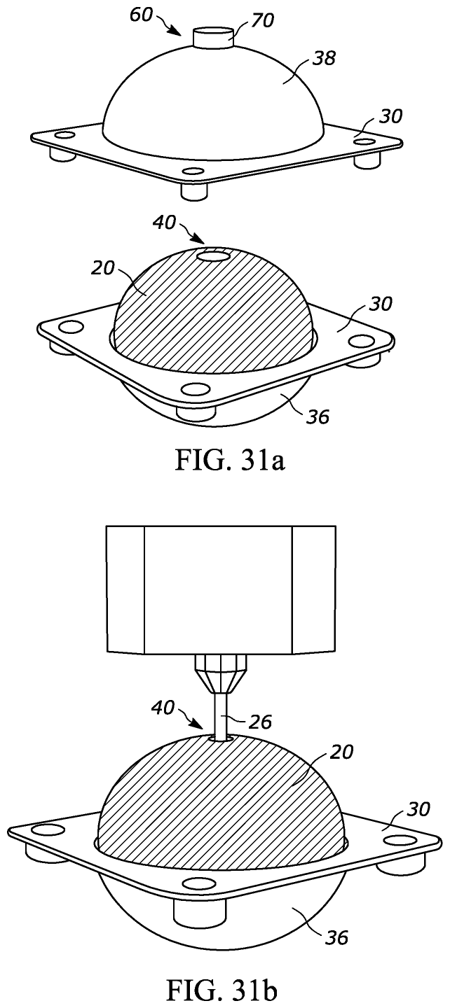

As shown in FIGS. 31a-31b, in another embodiment the entire upper portion (38) of the preformed mold (30) may be removed prior to dispensing the inner material (26) into the inner cavity (24) of the shell (20).

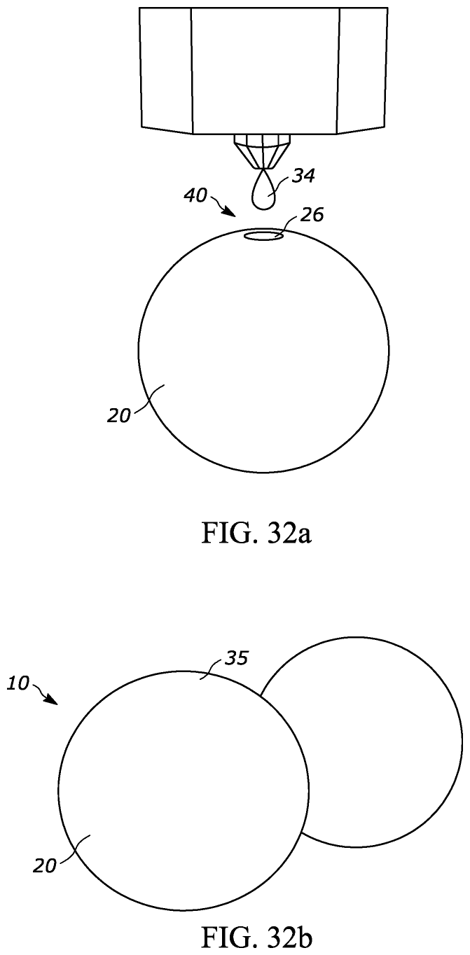

As shown in FIGS. 32a-32b, cannabis-derived resin (34) is then dispensed into the open channel (40) of the outer shell (20) and overtop the inner material (26) to create a seal (35) that fully encapsulates the inner material (26) within the outer shell (20). The preformed mold (30) may be removed before or after the seal (35) is applied.

One aspect (“aspect one”) relates to a dosing capsule comprising a shell, an inner material and a seal; the shell comprising a cannabis-derived resin; and the inner material comprising an ingestible material, whereby the shell and seal substantially encapsulate the inner material.

Another aspect (“aspect two”) relates to the capsule of aspect one, wherein the seal comprises a cannabis-derived resin.

Another aspect (“aspect three”) relates to the capsule of aspect two, wherein the cannabis-derived resin comprising the shell is in a hardened state at ambient temperature, and when the capsule is heated to a pre-determined temperature, the shell liquefies, thereby allowing the inner material to be ingested.

Another aspect (“aspect four”) relates to the capsule of aspect one, wherein the shell comprises a cannabis-derived resin that is derived from cannabis by a solventless extraction method.

Another aspect (“aspect five”) relates to the capsule of aspect one, wherein the shell comprises a cannabis-derived resin that is derived from cannabis by a solvent extraction method.

Another aspect (“aspect six”) relates to the capsule of aspect one, wherein the cannabis-derived resin is selected from the group consisting of THCA, CBDA and CBGA.

Another aspect (“aspect seven”) relates to the capsule of aspect two, wherein the cannabis-derived resin used for the seal is selected from the group consisting of THCA, CBDA and CBGA.

Another aspect (“aspect eight”) relates to the capsule of aspect one, wherein the inner material comprises a material selected from the group consisting of marijuana concentrates, rosin, resin, water hash, THCA, hash, flower, oils, hemp, CBD, liquid nicotine, terpenes and lavender.

Another aspect (“aspect nine”) relates to the capsule of aspect two, wherein the shell begins to liquefy at a temperature of about 200-220 degrees Fahrenheit.

Another aspect (“aspect ten”) relates to the capsule of aspect one, wherein the shell is shaped substantially semi-spherical.

Another aspect (“aspect eleven”) relates to the capsule of aspect two, wherein the shell begins to liquefy at a temperature of about 98.6 degrees Fahrenheit.

Another aspect (“aspect twelve”) relates to the capsule of aspect one, wherein the shell has a thickness of between about 60-600 microns.

One aspect (“aspect thirteen”) relates to a method of forming a cannabis-derived resin outer shell of a dosing capsule comprising the steps of: providing a pulverized cannabis-derived resin in crystalline powder form; providing a pre-formed mold; placing the cannabis-derived resin into the lower portion of the pre-formed mold; heating the pre-formed mold containing the cannabis-derived resin to a pre-determined temperature, thereby allowing the cannabis-derived resin to liquefy; pressing the upper portion of the pre-formed mold into the lower portion containing the liquefied cannabis-derived resin, thereby allowing the cannabis-derived resin to assume the shape of the mold; allowing the mold to cool; removing the top portion of the pre-formed mold.

Another aspect (“aspect fourteen”) relates to the method of aspect thirteen, wherein the cannabis-derived resin begins to liquefy at a temperature of about 200-220 degrees Fahrenheit.

Another aspect (“aspect fifteen”) relates to the method of aspect thirteen, wherein the cannabis-derived resin is THCA (tetrahydrocannabinol acid).

Another aspect (“aspect sixteen”) relates to the method of aspect thirteen, wherein the cannabis-derived resin is CBDA (cannabidiolic acid).

Another aspect (“aspect seventeen”) relates to the method of aspect thirteen, wherein the cannabis-derived resin is selected from the group consisting of THCA, CBDA and CBGA.

Many different arrangements of the process of making and components described above, as well as components and steps not shown, are possible without departing from the spirit and scope of the present disclosure. The aforementioned method has been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

I claim:

1. A dosing capsule, comprising:
a shell, an inner material and a seal;
the shell comprising a cannabis-derived resin;
and the inner material comprising an ingestible material,
whereby the shell and seal substantially encapsulate the inner material.

2. The capsule of claim 1 wherein the seal comprises a cannabis-derived resin.

3. The capsule of claim 2 wherein:
the cannabis-derived resin comprising the shell is in a hardened state at ambient temperature, and when the capsule is heated to a pre-determined temperature, the shell liquefies, thereby allowing the inner material to be ingested.

4. The capsule of claim 1 wherein the shell comprises a cannabis-derived resin that is derived from cannabis by a solventless extraction method.

5. The capsule of claim 1 wherein the shell comprises a cannabis-derived resin that is derived from cannabis by a solvent extraction method.

6. The capsule of claim 1 wherein the cannabis-derived resin is selected from the group consisting of THCA, CBDA and CBGA.

7. The capsule of claim 2, wherein the cannabis-derived resin used for the seal is selected from the group consisting of THCA, CBDA and CBGA.

8. The capsule of claim 1 wherein the inner material comprises a material selected from the group consisting of marijuana concentrates, rosin, resin, water hash, THCA, hash, flower, oils, hemp, CBD, liquid nicotine, terpenes and lavender.

9. The capsule of claim 2 wherein the shell begins to liquefy at a temperature of about 200-220 degrees Fahrenheit.

10. The capsule of claim 1 wherein the shell is shaped substantially semi-spherical.

11. The capsule of claim 2 wherein the shell begins to liquefy at a temperature of about 98.6 degrees Fahrenheit.

12. The capsule of claim 1 wherein the shell has a thickness of between about 60-600 microns.

13. The method of forming a cannabis-derived resin outer shell of a dosing capsule comprising the steps of:
providing a pulverized cannabis-derived resin in crystalline powder form;
providing a pre-formed mold;
placing the cannabis-derived resin into the lower portion of the pre-formed mold;
heating the pre-formed mold containing the cannabis-derived resin to a pre-determined temperature, thereby allowing the cannabis-derived resin to liquefy;
pressing the upper portion of the pre-formed mold into the lower portion containing the liquefied cannabis-derived resin, thereby allowing the cannabis-derived resin to assume the shape of the mold;
allowing the mold to cool;
removing the top portion of the pre-formed mold.

14. The method of claim 13 wherein the cannabis-derived resin begins to liquefy at a temperature of about 200-220 degrees Fahrenheit.

15. The method of claim 13 wherein the cannabis-derived resin is THCA (tetrahydrocannabinol acid).

16. The method of claim 13 wherein the cannabis-derived resin is CBDA (cannabidiolic acid).

17. The method of claim 13 wherein the cannabis-derived resin is selected from the group consisting of THCA, CBDA and CBGA.
